(19) United States
(12) Patent Application Publication
(10) Pub. No.: US 2021/0268405 A1
(43) Pub. Date: Sep. 2, 2021

(54) METHOD FOR PRODUCING RECOMBINANT ADENOVIRUS

(71) Applicant: SINOSHENG (SHENZHEN) GENE INDUSTRY DEVELOPMENT CO., LTD., Shenzhen,Guangdong (CN)

(72) Inventors: Wei XU, Shenzhen, Guangdong (CN); Hong LIN, Shenzhen, Guangdong (CN)

(21) Appl. No.: 17/256,458

(22) PCT Filed: Aug. 31, 2018
(86) PCT No.: PCT/CN2018/103656

§ 371 Date: Dec. 28, 2020

(30) Foreign Application Priority Data
Jun. 28, 2018 (CN) 201810686129.0

(51) Int. Cl. B01D 15/36; C12N 7/02

(52) U.S. Cl. CPC B01D 15/361; C12N 2710/10021; C12N 7/02

(57) ABSTRACT

The present application provides a method for producing recombinant adenovirus. The method comprises expansion of the recombinant adenovirus packaging cells from the Working Cell Bank and re-expansion of the cells after inoculation in a packed bed bioreactor in order to obtain a re-expanded recombinant adenovirus. The present application also provides the use of a packed bed bioreactor for recombinant adenovirus production.

### BACKGROUND

### Technical Field

The present invention belongs to the technical field of biological medicine, and relates to a method for producing recombinant adenovirus, in particular to a method and process for mass production of recombinant adenovirus gene therapy product by GMP.

### Background Art

After decades of research and development, the gene therapy has achieved considerable development and a breakthrough has been made in product development. Sibiono GeneTech Co. Ltd., Shenzhen's Gendicine recombinant adenovirus p53 product, UniQure's Glybera recombinant adeno-associated virus (AAV) product and Spark Therapeutics' Luxturna recombinant adeno-associated virus (AAV) product have been marketed with the approval of the Drug Regulatory Administration, bringing benefits to the patients and the society. The traditional method for producing gene therapy products in the laboratory is no longer able to meet the requirements for the mass production of gene therapy products by GMP. There is an urgent need to develop a cost-effective method and process that meet the technical requirements of GMP and can be used for mass production.

### SUMMARY OF THE INVENTION

The present application provides a method for producing recombinant adenovirus. The method comprises expansion of the recombinant adenovirus packaging cells from the Working Cell Bank and re-expansion of the cells after inoculation in a packed bed bioreactor in order to obtain a re-expanded recombinant adenovirus.

The present application also provides the use of a packed bed bioreactor for recombinant adenovirus production.

### BRIEF DESCRIPTION OF THE DRAWINGS

FIG. 1 shows a flow diagram of the method and process for mass production of the recombinant adenovirus.

FIG. 2 shows the structure (a) and working principle (b) of a packed bed bioreactor (e.g., CelliGen plus, New Brunswick Scientific, Co. Inc. USA), wherein (A) culture medium inlet; (B) harvesting outlet; (C) adjustable harvest leveling tube; (D) cell-free culture medium; (E) saturates the disk bed by aerated culture medium; (F) filtered bubbles in the aeration chamber; (G) draft tube; (H) disk bed basket; (I) PBS harvesting; (J) air inlet; (K) bearing box; (L) impeller port; (M) distributor pipeline; (N) water coat; (O) polyester disk; (P) cell-free effluent; (Q) culture medium calculation; (R) water coat outlet; (S) water coat inlet.

FIG. 3(a) and FIG. 3(b) show HD structure of Fibra-Cel Disks.

FIG. 4 shows a flow diagram of production in an embodiment disclosed in the present application.

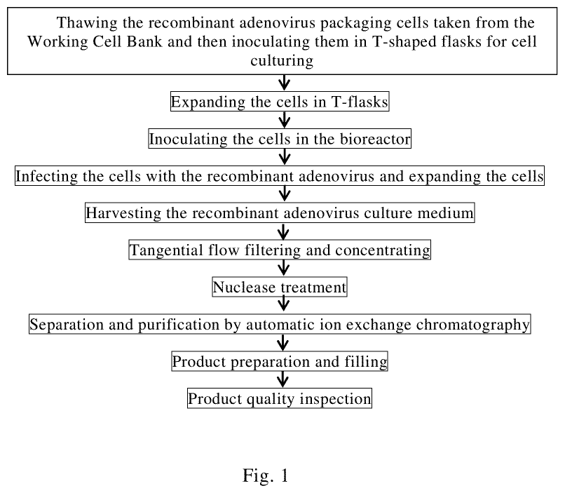

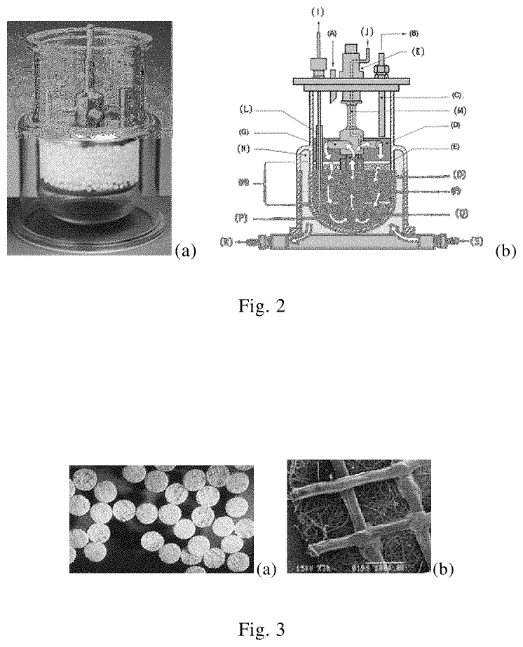

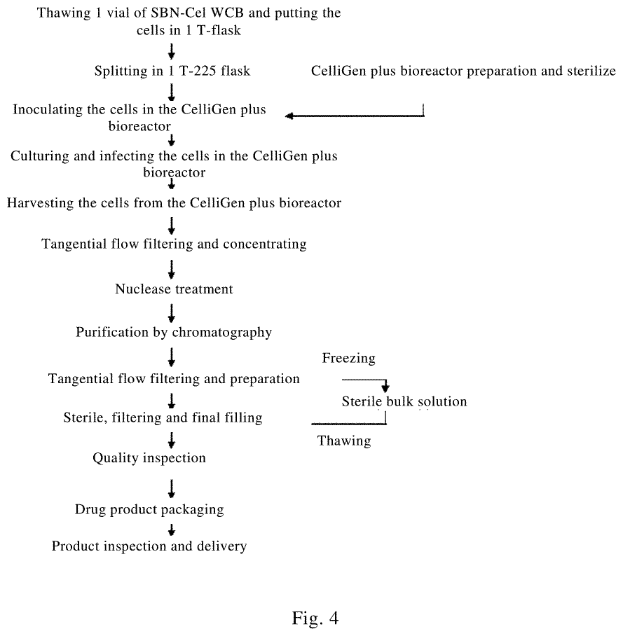

### EMBODIMENTS

In one embodiment of the present application, a method for producing recombinant adenovirus is provided. The method comprises expansion of the recombinant adenovirus packaging cells from the Working Cell Bank and re-expansion of the cells after inoculation in a packed bed bioreactor in order to obtain re-expanded recombinant adenovirus.

In one embodiment, the packed bed bioreactor is CelliGen plus (New Brunswick Scientific, Co. Inc. USA or iCELLis (Pall Corporation, USA).

In one embodiment, the cells for recombinant adenovirus production are clone cells with adherent growth characteristics.

In one embodiment, the cells for recombinant adenovirus production are SBN-1 293 clone cells with adherent growth characteristics.

In one embodiment, the fillers for the packed bed bioreactor include polyester and polypropylene.

In one embodiment, the fillers for the packed bed bioreactor include a polyester mesh and a polypropylene support frame.

In one embodiment, the method also comprises the treatment of the obtained recombinant adenovirus by one or more of the following steps: filtering, concentrating, nuclease treatment and purification by ion exchange chromatography.

In one embodiment, a tangential flow microfiltration and/or a tangential flow ultrafiltration system is used to filter the recombinant adenovirus solution.

In one embodiment, the recombinant adenovirus is purified by ion exchange chromatography.

In one embodiment, Sepharose Q XL resin or Source Q resin is used to purify the recombinant adenovirus.

In one embodiment of the present application, the use of a packed bed bioreactor for recombinant adenovirus production is provided.

In one embodiment, a method suitable for mass production of recombinant adenovirus gene therapy product is provided, successfully realizing the industrialization of the recombinant adenovirus p53 product.

In one embodiment, an improved method for mass production of recombinant adenovirus is provided. A bioreactor and automatic column chromatography are used for separation and purification in the improved method, realizing more economical and effective mass production of recombinant adenovirus product by GMP.

In one embodiment, the production method comprises the following main steps: cell expansion; bioreactor inoculation, culture medium perfusion and cell re-expansion; cell infection with the recombinant adenovirus and expansion; recombinant adenovirus culture medium harvesting, filtering and concentrating; nuclease treatment; separation and purification by automatic ion exchange chromatography; product preparation and filling.

In one embodiment, the method provided by the present application has good amplification characteristics, and therefore can meet the various technical requirements of the Drug Regulatory Administration for production by GMP, fully ensuring the quality and biological activity of the product.

In one embodiment, the present invention provides a method for mass production of a recombinant adenovirus. A packed bed bioreactor is used upstream for 3D high-concentration cell culture while adherently growing cells are infected with the recombinant adenovirus and expanded. Compared with 2D adherent cell culture and suspended cell culture, the 3D high-concentration cell culture method is more efficient and suitable for production by GMP. Column chromatography is used downstream for separation and purification, realizing more economical and effective mass production of a recombinant adenovirus production by GMP for the clinical treatment of cancer patients. The production method provided by the present application comprises the following main steps: cell expansion; packed bed bioreactor inoculation; culture medium perfusion and cell re-expansion; cell infection with the recombinant adenovirus and expansion; recombinant adenovirus culture medium harvesting, filtering and concentrating; nuclease treatment; separation and purification by automatic ion exchange chromatography; product preparation and filling. The method has good amplification characteristics and cost-effectiveness, and can meet the various technical requirements of the Drug Regulatory Administration for production by GMP, fully ensuring the quality and biological activity of the product.

Drawing 1 shows a flow diagram of the method and process for mass production of the recombinant adenovirus in one embodiment of the present application.

In one embodiment, the method provided by the present invention comprises the following steps (all operations are performed in a GMP-specified clean room):

### Cell Expansion

About 5×10⁶˜1×10⁷ recombinant adenovirus packaging cells are taken from the Working Cell Bank. After being unfrozen, the cells are inoculated in a T-flask for cell culture. The inoculation density is about 0.5×10⁴˜5×10⁴/cm². Then, the cells are cultured in an incubator with 5% CO₂ at 37° C. until the cell density rises to about 90%. Trypsin-EDTA is used for cell harvesting, with the cells inoculated in more T-flasks for cell expansion. The inoculation density remains at 0.5×10⁴˜5×10⁴/cm². Cell expansion is repeated until there are enough cells that can be used for bioreactor inoculation.

### Bioreactor Inoculation, Culture Medium Perfusion and Cell Re-Expansion

The present invention adopts a packed bed bioreactor (e.g., CelliGen plus, New Brunswick Scientific, Co. Inc. USA) for large-scale cell culture. Drawing 2 shows the structure and working principle of the packed bed bioreactor.

About 250 g of Fibra-Cel Disks (New Brunswick Scientific, Co. Inc.) are packaged in the stainless steel basket of the cleansed packed bed bioreactor. Then, 10 L of PBS is added to the bioreactor. After rubber tube and bio-probe connection, the bioreactor is placed in a sterilizing cabinet and sterilized at 121° C. for 45 min.

The Fibra-Cel Disks adopted consist of polyester and polypropylene or consist of a polyester mesh and polypropylene support frame. Drawing 3(a) and Drawing 3(b) show the HD structure of Fibra-Cel Disks.

The cells grow the surface and internal voids of the Fibra-Cel Disks, ensuring high cell concentration.

The PBS solution is removed from the sterilized bioreactor first, and then 5 L of DMEM+10% FBS is added to the bioreactor. The cells harvested from the T-flask are inoculated in the bioreactor for culturing. The cell inoculation amount is about 1×10⁹˜1×10¹⁰. The pH value and dissolved oxygen (DO) value of the culture medium are automatically controlled by the controller in the bioreactor with the aid of the pH and DO probes. The pH value is controlled at 6.5-8.2, while the DO value is controlled at 15%-75%. The temperature of the culture medium is automatically controlled at 36° C.-37° C. through the circulating water in the water coat. The rotating agitator in the bioreactor is used to agitate the culture medium. The agitation rate is controlled at 50-200 rpm.

The cells are left to grow in the bioreactor for 5-10 days. On the 3^{rd} day after cell inoculation, culture medium perfusion is performed so as to continuously provide nutrients for the culture medium and remove harmful cell metabolites. The culture medium perfusion volume is adjusted in order that the glucose concentration of the culture medium in the bioreactor should not be less than 0.5 g/L. After 5 days of growth, the cells can be infected with the recombinant adenovirus.

### Cell Infection With the Recombinant Adenovirus and Expansion

The recombinant adenovirus is taken from the Working Virus Bank to infect the cells in the bioreactor. The multiplicity of infection (MOI) is controlled at 10-100 vp/cell. Then, the cells are cultured for 7 more days. 3-5 days after virus infection, the recombinant adenovirus will be released into the culture medium. At this point, it is time to start harvesting the culture medium containing the recombinant adenovirus until the end of the culture process. The culture medium containing the recombinant adenovirus harvested every day is stored temporarily at 4° C. for final unified purification.

### Filtering and Concentrating of Recombinant Adenovirus Culture Medium

First, all the harvested recombinant adenovirus culture medium is filtered through the tangential flow microfiltration system of 0.65 μm to remove large impurities such as cell debris. Then, the filtered medium is concentrated by about 10-50 times through another 300 KD MWCO tangential flow ultrafiltration system. The concentrated medium is percolated and then poured into another buffer for subsequent nuclease treatment and separation and purification by ion exchange column chromatography. The percolation coefficient is 3-10. The pressure parameters are controlled at 5-20 psi during the tangential flow ultrafiltration concentration and percolation. A buffer can be used to dilute the concentrated and percolated medium before nuclease is added so as to increase the efficiency of nuclease treatment.

### Nuclease Treatment

Benzonase™ is used in this process. It is a broad-spectrum endonuclease widely used in the production process of biological products. Benzonase™ is added to the concentrated and percolated medium diluted with the buffer to degrade free nucleic acid molecules. The dosage is set to 10-20 u/mL. Degradation lasts 1 h in a water bath at 37° C.

### Separation and Purification by Automatic Ion Exchange Column Chromatography

The recombinant adenovirus culture medium treated with Benzonase™ is separated and purified by anion exchange column chromatography at room temperature. An automatic purification system controlled by the software Unicorn is used for purification. The anion exchange resin (e.g., Sepharose Q XL) is packed in the BPG300 chromatographic column. The packing volume is about 2.5 L. Before it is used, the chromatographic column undergoes an HETP (Height Equivalent to Theoretical Plate) test to ensure that it has good performance. Before sample loading, 1.0N NaOH solution is used to disinfect the purification system and the chromatographic column loaded with anion exchange resin. After being treated, the chromatographic column is first equilibrated with buffer A (20 mM Tris, pH 8.0). After the equilibration of the chromatographic column, the recombinant adenovirus culture medium starts being loaded. After loading, the chromatographic column is washed by buffer A for around 5-8 CV (column volume) until the UV absorption value decreases to the baseline. Soon afterwards, the recombinant adenovirus absorbed on the chromatographic column is eluted by a 30 CV linear NaCl gradient from buffer A to buffer B (20 mMTris, pH 8.0, 2M NaCl). During the elution process, the software Unicorn is used for automatic monitoring over the UV absorption value at 260 nm to determine where the recombinant adenovirus reaches a peak. When the UV absorption value at 260 nm rises to 0.1 AU, the recombinant adenovirus corresponding to the purified recombinant adenovirus peak is automatically received into a sterile flask. When the UV absorption value at 260 nm declines to 0.2 AU, reception is stopped. Then, the chromatographic column and purification system are washed with alkali liquor, buffer and acid fluid in sequence. Finally, the chromatographic column and purification system are stored in 0.1 N NaOH solution.

### Product Preparation and Filling

First of all, after purification by column chromatography, the recombinant adenovirus is further concentrated with a small 300 KD MWCO tangential flow ultrafiltration system (such as the Millipore Pellicon system) to meet the requirements for the virus titer (e.g., 1×10¹² vp/mL). Then, it is further percolated with a confecting solution of the recombinant adenovirus product (such as 20 mMTris-HCl, pH 8.0, containing 10% glycerol). The percolation coefficient is set to 10-15 to ensure that the purified recombinant adenovirus is completely transferred into the confecting solution of the product and that the product has high stability. The pressure parameters are controlled at 5-10 psi during the tangential flow ultrafiltration concentration and percolation. After being filtered with a 0.22 μm sterile filter, the recombinant adenovirus now can be used as sterile bulk solution. A sample of the sterile bulk solution is taken for quality inspection. The sterile bulk solution can be stored temporarily in a freezer at −80° C. Then, the qualified sterile bulk solution is bottled as a recombinant adenovirus drug preparation.

One or more batches of sterile bulk solution to be bottled are left aside for a whole night to be unfrozen at room temperature. Then, the 0.22 μm sterile filter is used again to filter the sterile bulk solution before it is bottled. After a sample of the sterile solution is taken for quality inspection, an automatic filling machine is used to bottle the sterile solution and cap the bottles. After the end of filling, samples of the recombinant adenovirus product are taken for quality inspection. The qualified solution can be used in clinical practice with the approval of Quality Department.

### EMBODIMENTS

### Embodiment 1: Production of a Recombinant Adenovirus Anticancer Injection

### 1. Cell Expansion

About 5×10⁶ cells were taken from the Working Cell Bank of recombinant adenovirus. After thawing treatment, the cells were inoculated in a T-flask for cell culturing. The inoculation density was about 0.5×10⁴˜5×10⁴/cm². Then, the cells were cultured in an incubator with 5% CO₂ at 37° C. until the cell density rises to about 90%. Trypsin-EDTA was used for cell harvesting, with the cells inoculated in more T-flasks for cell expansion. The inoculation density remained at 0.5×10⁴˜5×10⁴/cm². Cell expansion was repeated until there are about 1×10⁹ cells that could be used for bioreactor inoculation.

### 2. Cell Inoculation and Infection With Virus in Celligen Reactor

2.1 2×10⁹ cells were inoculated in a packed bed bioreactor containing 5 L of DMEM+10% FBS for culturing. The pH was controlled at 6.5-8.2, while the DO value was controlled at 15%-75%. The temperature of the culture solution was automatically controlled at 36° C.-37° C. through the circulating water in the water coat. The rotating agitator in the bioreactor was used to agitate the culture solution. The agitation rate was controlled at 50-200 rpm. On the 3^{rd} day after cell inoculation, culture solution perfusion was performed so as to continuously provide nutrients for the culture solution and remove harmful cell metabolites. The culture solution perfusion volume was adjusted in order that the glucose concentration of the culture solution in the bioreactor should not be less than 0.5 g/L. After 5 days of growth, the cells could be infected with the recombinant adenovirus.

2.2 The recombinant adenovirus was taken from the Working Virus Bank to infect the cells in the bioreactor. The multiplicity of infection (MOI) was controlled at 10-100 vp/cell. Then, the cells were cultured for 7 more days. 3-5 days after virus infection, the recombinant adenovirus was released into the culture solution. At this point, the culture medium containing the recombinant adenovirus began to be harvested until the end of the culture process. 35 L was harvested, with the viral load of 1.6×10¹⁵ vp. The culture medium containing the recombinant adenovirus harvested every day was stored temporarily at 4° C. for final unified purification.

### 3. Primary Purification and Further Purification of Recombinant Adenovirus Culture Medium

First, all the harvested recombinant adenovirus culture medium was filtered through the tangential flow microfiltration system of 0.65 μm to remove large impurities such as cell debris. Then, the filtered medium was concentrated to 3 L through another 300 KD MWCO tangential flow ultrafiltration system, with the total virus content of 1×10¹⁵ vp. Benzonase™ was added to the concentrated and percolated medium diluted with the buffer to degrade free nucleic acid molecules. The recombinant adenovirus culture medium treated with Benzonase™ was separated and purified by anion exchange column chromatography at room temperature. The anion exchange resin (e.g., Sepharose Q XL) was packed in the BPG300 chromatographic column. The packing volume was about 2.5 L. Before it was used, the chromatographic column underwent an HETP (Height Equivalent to Theoretical Plate) test to ensure that it had good performance. Before sample loading, 1.0N NaOH solution was used to disinfect the purification system and the chromatographic column loaded with anion exchange resin. After being treated, the chromatographic column was first equilibrated with buffer A (20 mM Tris, pH 8.0). After the equilibration of the chromatographic column, the recombinant adenovirus culture medium started being loading. After loading, the chromatographic column is washed by buffer A for 5 CV (column volume) until the UV absorption value decreases to the baseline. Soon afterwards, the recombinant adenovirus absorbed on the chromatographic column is eluted by a 30 CV linear NaCl gradient from buffer A to buffer B (20 mMTris, pH 8.0, 2M NaCl). During the elution process, the software Unicorn was used for automatic monitoring over the UV absorption value at 260 nm to determine where the recombinant adenovirus reached a peak. When the UV absorption value at 260 nm rose to 0.1 AU, the recombinant adenovirus corresponding to the purified recombinant adenovirus peak was automatically received into a sterile flask. When the UV absorption value at 260 nm declined to 0.2 AU, reception was stopped. At this time, the virus culture medium volume was 600 ml, the virus concentration was 1×10¹² vp/ml, the virus purity was 95%, and the total viral load was 6×10¹⁴ vp. Then, the chromatographic column and purification system were washed with alkali liquor, buffer and acid fluid in sequence.

### 4. Product Preparation and Filling

Then, the solution was further percolated with a confecting solution (20 mMTris-HCl, pH 8.0, containing 10% glycerol). The percolation coefficient was set to 10-15 to ensure that the purified recombinant adenovirus was completely transferred into the confecting solution of the product and that the product had high stability. After being filtered with a 0.22 μm sterile filter, the recombinant adenovirus now can be used as sterile bulk solution 24 ml sterile bulk solution was taken as a sample for quality inspection, including sterility testing, bacterial endotoxin testing and virus concentration testing. The sterile bulk solution could be stored temporarily in a freezer at −80° C. Then, the qualified sterile bulk solution was bottled as a recombinant adenovirus drug preparation.

A batch of sterile bulk solution was left aside for a whole night to be unfrozen at room temperature. Then, the 0.22 μm sterile filter was used again to filter the sterile bulk solution before it was bottled. After a sample of the sterile solution was taken for quality inspection, an automatic filling machine was used to bottle the sterile solution and cap the bottles. After the end of filling, samples of the recombinant adenovirus product were taken for quality inspection. The test items are as follows: sterility, appearance (light white liquid, free of visible particles), endotoxin<10 EU/piece, with the culture method and fluorescent staining used to test the mycoplasma negative, virus concentration 1×10¹² vp/vial, virus purity≥95%, virus activity≥3.3×10¹⁰ IU/vial, specific activity≥3.3%, biologically active cancer cell TCID50: 100 to 500 MOI, actively replicated virus<1 RCA/3×10¹⁰ vp, residual cell protein≤100 ng/vial, residual bovine serum protein≤50 ng/vial, residual cell DNA≤10 ng/vial, residual Benzonase™≤1 ng/vial, free of AAV pollution, no abnormal toxicity, without particulate matter and with proper osmotic pressure molar concentration.

### Embodiment 2: Mass Production of a Recombinant Adenovirus Anticancer Injection by GMP

### 1. Cell Expansion

About 9.8×10⁶ cells were taken from the Working Cell Bank of recombinant adenovirus. After being unfrozen, the cells were inoculated in a T-flask for cell culture. The inoculation density was about 0.5×10⁴˜5×10⁴/cm². Then, the cells were cultured in an incubator with 5% CO2 at 37° C. until the cell density rose to about 90%. Trypsin-EDTA was used for cell harvesting, with the cells inoculated in more T-flasks for cell expansion. The inoculation density remained at 5×10⁴/cm². Cell expansion is repeated until there were about 2.5×10⁹ cells that could be used for bioreactor inoculation.

### 2. Cell Inoculation and Infection With the Virus in Celligen Reactor

2.1 2.5×10⁹ cells were inoculated in a packed bed bioreactor containing 15 L of DMEM+10% FBS for culturing. The pH was controlled at 6.5-8.2, while the DO value was controlled at 15%-75%. The temperature of the culture solution was automatically controlled at 36° C.-37° C. through the circulating water in the water coat. The rotating agitator in the bioreactor is used to agitate the culture solution. The agitation rate is controlled at 50-200 rpm. On the 3^{rd} day after cell inoculation, culture solution perfusion was performed so as to continuously provide nutrients for the culture solution and remove harmful cell metabolites. The culture solution perfusion volume was adjusted in order that the glucose concentration of the culture solution in the bioreactor should not be less than 0.5 g/L. After 9 days of growth, the cells could be infected with the recombinant adenovirus.

2.2 The recombinant adenovirus was taken from the Working Virus Bank to infect the cells in the bioreactor. The multiplicity of infection (MOI) was controlled at 10-100 vp/cell. Then, the cells are cultured for 6 more days. 3-5 days after virus infection, the recombinant adenovirus was released into the culture solution. At this point, the culture medium containing the recombinant adenovirus began to be harvested until the end of the culture process. 75 L was harvested, with the viral load of 3×10¹⁵ vp. The culture medium containing the recombinant adenovirus harvested every day is stored temporarily at 4° C. for final unified purification.

### 3. Primary Purification and Further Purification of Recombinant Adenovirus Culture Medium

First, all the harvested recombinant adenovirus culture medium was filtered through the tangential flow microfiltration system of 0.65 μm to remove large impurities such as cell debris. Then, the filtered medium was concentrated to 6 L through a 300 KD MWCO tangential flow ultrafiltration system, with the total virus content of 2.7×10¹⁵ vp. Benzonase™ was added to the concentrated and percolated medium diluted with the buffer to degrade free nucleic acid molecules. The recombinant adenovirus culture medium treated with Benzonase™ was separated and purified by anion exchange column chromatography at room temperature. The anion exchange resin (e.g., Sepharose Q XL) was packed in the BPG300 chromatographic column. The packing volume was about 2.5 L. Before it was used, the chromatographic column underwent an HETP (Height Equivalent to Theoretical Plate) test to ensure that it had good performance. Before sample loading, 1.0N NaOH solution was used to disinfect the purification system and the chromatographic column loaded with anion exchange resin. After being treated, the chromatographic column was first equilibrated with buffer A (20 mM Tris, pH 8.0). After the equilibration of the chromatographic column, the recombinant adenovirus culture medium started being loading. After loading, the chromatographic column is washed by buffer A for 5 CV (column volume) until the UV absorption value decreases to the baseline. Soon afterwards, the recombinant adenovirus absorbed on the chromatographic column is eluted by a 30 CV linear NaCl gradient from buffer A to buffer B (20 mMTris, pH 8.0, 2M NaCl). During the elution process, the software Unicorn was used for automatic monitoring over the UV absorption value at 260 nm to determine where the recombinant adenovirus reached a peak. When the UV absorption value at 260 nm rose to 0.1 AU, the recombinant adenovirus corresponding to the purified recombinant adenovirus peak was automatically received into a sterile flask. At this time, the virus culture medium volume was 2,500 ml, the virus concentration was 90×10¹⁰ vp/ml˜95×10¹⁰ vp/ml, the virus purity was 95%, and the total viral load was 2.2×10¹⁵ vp. Then, the chromatographic column and purification system were washed with alkali liquor, buffer and acid fluid in sequence.

### 4. Product Preparation and Filling

Then, the solution was further percolated with a confecting solution (20 mMTris-HCl, pH 8.0, containing 10% glycerol). The percolation coefficient was set to 10-15 to ensure that the purified recombinant adenovirus was completely transferred into the confecting solution of the product and that the product had high stability. After being filtered with a 0.22 μm sterile filter, the recombinant adenovirus now can be used as sterile bulk solution 24 ml sterile bulk solution was taken as a sample for quality inspection, including sterility testing, bacterial endotoxin testing and virus concentration testing. The sterile bulk solution could be stored temporarily in a freezer at −80° C. Then, the qualified sterile bulk solution was bottled as a recombinant adenovirus drug preparation.

A batch of sterile bulk solution was left aside for a whole night to be unfrozen at room temperature. Then, the 0.22 μm sterile filter was used again to filter the sterile bulk solution before it was bottled. After a sample of the sterile solution was taken for quality inspection, an automatic filling machine was used to bottle the sterile solution and cap the bottles. After the end of filling, samples of the recombinant adenovirus product were taken for quality inspection. The test items are as follows: sterility, appearance (light white liquid, free of visible particles), endotoxin<10 EU/vial, with the culture method and fluorescent staining used to test the mycoplasma negative, virus concentration 1×10¹² vp/vial, virus purity≥95%, virus activity≥3.3×10¹⁴ U/vial, specific activity≥3.3%, biologically active cancer cell TCID50: 100 to 500 MOI, actively replicated virus<1 RCA/3×10¹⁰ vp, residual cell protein≤100 ng/vial, residual bovine serum protein≤50 ng/vial, residual cell DNA≤10 ng/vial, residual Benzonase™≤1 ng/piece, free of AAV pollution, no abnormal toxicity, without particulate matter and with proper osmotic pressure molar concentration.

### INDUSTRIAL APPLICABILITY

The present application provides a method for producing recombinant adenovirus. The method comprises expansion of the recombinant adenovirus packaging cells from the Working Cell Bank and re-expansion of the cells after inoculation in a packed bed bioreactor in order to obtain a re-expanded recombinant adenovirus. The method has good amplification characteristics and cost-effectiveness, and can meet the various technical requirements of the Drug Regulatory Administration for production by GMP, fully ensuring the quality and biological activity of the product. The present application also provides the use of a packed bed bioreactor for recombinant adenovirus production. The method has bright industrial application prospects.

1. A method for producing recombinant adenovirus, comprising:
expanding recombinant adenovirus packaging cells from the Working Cell Bank and re-expanding of the recombinant adenovirus packaging cells after inoculation in a packed bed bioreactor in order to obtain a re-expanded recombinant adenovirus.

2. The method for producing recombinant adenovirus as claimed in claim 1, wherein the recombinant adenovirus packaging cells for recombinant adenovirus production are clone cells with adherent growth characteristics.

3. The method for producing recombinant adenovirus as claimed in claim 1, wherein the recombinant adenovirus packaging cells for recombinant adenovirus production are SBN-1 293 clone cells with adherent growth characteristics.

4. The method for producing recombinant adenovirus as claimed in claim 1, wherein fillers for the packed bed bioreactor include polyester and polypropylene.

5. The method for producing recombinant adenovirus as claimed in claim 1, wherein the method also comprises treating the re-expanded recombinant adenovirus by one or more of the following steps: filtering, concentrating, nuclease treatment, or purification by ion exchange chromatography.

6. The method for producing recombinant adenovirus as claimed in claim 5, wherein at least one of a tangential flow microfiltration or a tangential flow ultrafiltration system is used to filter the re-expanded recombinant adenovirus during the filter.

7. The method for producing recombinant adenovirus as claimed in claim 5, wherein the re-expanded recombinant adenovirus is purified by ion exchange chromatography.

8. The use of a packed bed bioreactor for recombinant adenovirus production.

9. The use as claimed in claim 8, wherein cells for recombinant adenovirus production are clone cells with adherent growth characteristics.

10. The use as claimed in claim 9, wherein fillers for the packed bed bioreactor include polyester and polypropylene.

11. The method for producing recombinant adenovirus as claimed in claim 1, wherein fillers for the packed bed bioreactor include polyester mesh and a polypropylene support frame.

12. The use as claimed in claim 8, wherein cells for recombinant adenovirus production are SBN-1 293 clone cells with adherent growth characteristics.

13. The use as claimed in claim 9, wherein fillers for the packed bed bioreactor include a polyester mesh and a polypropylene support frame.

14. The method for producing recombinant adenovirus as claimed in claim 2, wherein the recombinant adenovirus packaging cells for recombinant adenovirus production are SBN-1 293 clone cells with adherent growth characteristics.

15. The method for producing recombinant adenovirus as claimed in claim 2, wherein fillers for the packed bed bioreactor include polyester and polypropylene.

16. The method for producing recombinant adenovirus as claimed in claim 3, wherein fillers for the packed bed bioreactor include polyester and polypropylene.

17. The method for producing recombinant adenovirus as claimed in claim 2, wherein the method also comprises treating the re-expanded recombinant adenovirus by one or more of the following steps: filtering, concentrating, nuclease treatment, or purification by ion exchange chromatography.

18. The method for producing recombinant adenovirus as claimed in claim 3, wherein the method also comprises treating the re-expanded recombinant adenovirus by one or more of the following steps: filtering, concentrating, nuclease treatment, or purification by ion exchange chromatography.

19. The method for producing recombinant adenovirus as claimed in claim 4, wherein the method also comprises treating the re-expanded recombinant adenovirus by one or more of the following steps: filtering, concentrating, nuclease treatment, or purification by ion exchange chromatography.

20. The method for producing recombinant adenovirus as claimed in claim 6, wherein the re-expanded recombinant adenovirus is purified by ion exchange chromatography.
